(19) United States
(12) Patent Application Publication
(10) Pub. No.: US 2013/0095044 A1
(43) Pub. Date: Apr. 18, 2013

(54) USE OF STREPTOCOCCUS SALIVARIUS IN THE TREATMENT OF CHRONIC INFECTIONS OF THE RESPIRATORY TRACT

(71) Applicant: D.M.G. ITALIA Srl, Pomezia (Rome) (IT)

(72) Inventors: Stefania STEFANI, Catania (CT) (IT); Licia TIBERI, Pomezia (Rome) (IT); Maria SANTAGATI, Catania (CT) (IT)

(73) Assignee: D.M.G. ITALIA Srl, Pomezia (Rome) (IT)

(21) Appl. No.: 13/644,357

(22) Filed: Oct. 4, 2012

Related U.S. Application Data
(63) Continuation-in-part of application No. PCT/IT2011/000104, filed on Apr. 7, 2011.

(30) Foreign Application Priority Data
Apr. 7, 2010 (IT) RM20IOA000163

(51) Int. Cl. A61K 35/74; C12N 1/20; A61K 31/19

(52) U.S. Cl. CPC A61K 35/74; A61K 31/19; C12N 1/20
USPC 424/45; 424/93.44; 435/253.4; 435/245

(57) ABSTRACT

A new microbial strain of the species Streptococcus salivarius for use in the treatment of inflammatory processes with or without infectious etiology. A further object of the present invention compositions including the strain and uses thereof.

The present invention provides a new microbial strain of the species Streptococcus salivarius for use in the treatment of inflammatory processes with or without infectious etiology. A further object of the present invention are compositions comprising said strain and uses thereof.

### STATE OF THE ART

Many of the ENT (Ear, Nose and Throat) diseases may originate from a fungal or bacterial infection in the upper tracts of the respiratory system; examples of such infections are some forms of otitis, sinusitis and/or nasal poliposis: usually the treatment of such forms is performed by using topical or oral antibiotics or anti-inflammatory agents.

Recently clinical studies have demonstrated that the administration of streptococci such as Streptococcus mitis, Streptococcus sanguinis, Streptococcus oralis in the form of spray to patients affected by Acute Otitis Media (AOM) interferes and/or inhibits the growth of pathogenic microorganisms responsible of the disease. However, these species of microorganisms have the serious disadvantage to be classified as potentially pathogenic species.

Recently the use of bacteria as a probiotic agent is continuously developing thanks to their capacity to maintain or restore the host's natural microbiome by interference with and/or inhibition of other microrganisms, mediated by antimicrobial peptide production such as bacteriocines.

More than 700 bacterial species are present in the oral cavity and, maintaining the bacterial communities unaltered, has a significant impact on general health, by either preventing or causing infections. Changes in the structure of this complex community could contribute to a shift in the balance of the resident microflora to a disease-associated species compositions (Aas et al., 2005; Caglar et al., 2005; Marsh et al., 1991).

Bacterial interference, such as antagonism, has a fundamental role in keeping the balance of the microbial ecology associated with the ability of bacterial species to interfere during surface colonization. This phenomenon represents an interesting mechanism of defence due to the capability of endogenous microflora to interfere or inhibit the growth of potential pathogens (Falagas et al., 2008).

In the oral cavity, Streptococcus salivarius, a non pathogenic and predominant colonizer in the oral microbiome, is one of the major bacteriocin producers which is able to coexist in the same environment and reduce the frequency of colonization of the main pathogens involved in the upper respiratory tract infections (Wescombe et al., 2009). For this reason S. salivarius is a good candidate for oral probiotic in humans. Probiotics are traditionally associated with gut health, many probiotics are used to prevent and treat several diseases mainly in the intestinal tract, and recently many studies have been involved in the development of oral probiotic applications. Clinical studies have demonstrated that the administration of streptococci such as Streptococcus mitis, Streptococcus sanguinis, Streptococcus oralis in the form of spray to patients affected by Acute Otitis Media (AOM) interferes and/or inhibits the growth of pathogenic microorganisms responsible of the disease. However, this species of microorganisms have the serious disadvantage to be classified as potentially pathogenic species.

The international patent application WO2004/072272 describes the use of a biologically pure culture of S. salivarius, isolated from patients oral cavity, in antibacterial compositions for the treatment of otitis media.

Walls et al. disclose that Streptococcus salivarius produces bacteriocin-like inhibitory substances (BLIS) which inhibit the activity of acute otitis media pathogens in the nasopharingeal floraof children and suggest that due to their low pathogenicity S. salivarius should be incorporated into trial of bacteriotherapy of recurrent AOM (Walls et al., 2003).

Recently Power et al. (Power et al. Eur J Clin Microbiol Infect Dis. 2008, 1261-3) have carried out preliminary studies on a group of children affected by AOM by administrating orally a pediatric composition comprising the strain S. salivarius K12. This strain has been previously used as a probiotic for oral hygiene and anti-halitosis.

The study carried out by Power and colleagues has revealed that only in a small percentage of the treated patients, the strain S. salivarius K12 has colonized the upper respiratory tract causing an improvement in symptoms of the disease being treated. The low capacity of the so far isolated strains of S. salivarius to colonize the upper respiratory tract makes less efficient their use in the adjuvant therapy against infections of the respiratory tracts.

So it was so strongly felt the need to isolate new non pathogenic strains that in addition to bactericide activity provide high capacity to colonize the respiratory tract.

### SUMMARY OF THE INVENTION

The inventors have succeeded in isolating from the nasopharynx of a healthy voluntary, a new bacterial strain belonging to the species S. salivarius deposited at the Institute Deutsche Sammlung von Mikroorganismen and Zellkulturen (DSMZ) under the filing number DSM 23307 in date 4 Feb. 2010.

The inventors, by in vitro experiments, show that this specific strain of Streptococcus salivarius is characterized by:
i) high inhibitory activity towards S. pneumoniae, stable in various culture conditions (BAC and TSYE);
ii) inhibitory activity towards particularly virulent and antibiotics multi-resistant serotypes responsible of invasive infections such as strain S. pneumoniae 19A;
iii) inhibitory activity towards S. pyogenes M-type 1;
iv) high adhesion capacity to the cells HEp-2 (epithelial cells of human carcinoma of the larynx) up to 57%;
v) absence of virulence genes;
vi) complete sensitivity to antibiotics.

Adhesion capacity of this strain to cells HEp-2, together with the properties not belonging to a pathogenic or potentially pathogenic species and producing bacteriocins able to inhibit the growth of S. pneumoniae and S. pyogenes, makes the strain of Streptococcus salivarius selected by the inventors and any other strain of Streptococcus salivarius with such features particularly suitable for treating bacterial and/or fungal infections of the upper respiratory tract. The utility of such organisms, that can be administered by pharmaceutical compositions, lies in their ability to colonize the respiratory tracts competing pathogenic species. It is therefore clear that adhesion ability of the administered strains to the HEp-2 type cells plays a key role for the efficacy of the same. The pattern of adhesion in vitro on cells derived from upper respiratory tract provides the adhesion and the retention of the bacteriocins producing strains.

Therefore, object of the present invention is a bacterial strain belonging to the Streptococcus salivarius species characterized by the ability to adhere to HEp-2 cells.

A further object of the invention is said bacterial strain as above defined and compositions comprising it for treating infections and/or inflammations of the upper respiratory tract.

Compositions comprising said bacterial strain and one or more carriers and/or diluents and/or excipients are object of the invention as well.

The advantages, features and the use modes of the present invention are evident from the following detailed description in some embodiments, presented as an example and without limitation.

### BRIEF DESCRIPTION OF THE DRAWINGS

FIG. 1 is showing bacterial adhesion to HEP-2 cell layer. Cell layers were observed after Giemsa staining using light microscopy. (1) Streptococcus salivarius K12, (2), Streptococcus salivarius 24SMB, (3) Streptococcus salivarius 4SMB, (4) negative control.

### DETAILED DESCRIPTION OF THE INVENTION

The present invention provides a new bacterial strain belonging to the species Streptococcus salivarius isolated by the inventors from the nosepharynx of a human voluntary subject; the strain has been identified by phenotypic and genotypic analysis.

The inventors have analyzed several nasal and pharyngeal swabs and several bacterial species have been isolated therefrom, but in one case only it has been isolated and selected a strain with the desired characteristics. The strain has a typical morphology of the S. salivarius species with a round shape of the colony and size of 1-2 mm in diameter, with entire and smooth margins. The bacterial strain can be grown on culture medium “Mitis salivarius” at 35° C., preferably in presence of 5% CO₂. The strain is able to adhere to HEp-2 cells and to inhibit the growth of the pathogen S. pneumoniae by bacteriocins production.

The strain has been called Streptococcus salivarius 24 SMBc and submitted in date 4 Feb. 2010 at the Institute Deutsche Sammlung von Mikroorganismen and Zellkulturen (DSMZ) GmbH, Braunschweig, Germany, under the filing number DSM 23307.

As already previously described the ability to adhere to HEp-2 cells makes this strain and even other strains belonging to the species Streptococcus salivarius having such feature particularly suitable for treating infections and/or inflammations of the upper respiratory tract, preferably for treating infections causing diseases such as acute otitis media, recurrence otitis media, nasal polyposis, sinusitis.

In the present description are defined as upper respiratory tracts the nasal and paranasal cavities, the pharynx, the larynx.

Object of the present invention are also compositions comprising strains of Streptococcus salivarius as above defined and one or more carriers, diluents and/or excipients.

Said compositions preferably comprise the bacterial strain Streptococcus salivarius filing number DSM 23307.

Bacteria can be in suspension, freeze-dryed or inactivated, provided they are not killed. The preparation of the compositions of the invention can then be implemented by freeze-drying of bacterial cultures, mixing freeze-dryed both in suspension with water or with further suitable excipients and optionally with addition of further active principles.

The amount of bacteria in said composition is preferably in the range between 10³ and 10¹⁰ CFU for each gram of composition.

Examples of excipients that can be used in such compositions are: rubber, xanthan, carboxylmethyl cellulose, silicone, Vaseline, white soft, magnesium stearate, maltodextrin, mannitol, starch, glucose, glycerine, propylene glycol, and similar.

Said compositions may include also carriers idoneous to improve the bioavailability, the stability and the endurance of the microrganism.

Said composition may comprise carriers to further improve the adhesion of the microorganism adhesion on the mucosal surface such as the EG56 polymer (Bis-Methoxy PEG-13 PEG-438/PPG-110 SMDI Copolymer), a heat-sensitive polymer able to increase the viscosity and thus the adhesiveness by increasing the temperature or Gantrex (PVM/MA Copolimer).

Said compositions may be in any form considered by the expert of the technical field suitable to be administered topically, orally, or through the respiratory tract.

For administration through the respiratory tract in the present description it has to be intended nasal or by inhalation administration.

Examples of suitable pharmaceutical forms are cream, lotion, gel, ointment, solution, suspension, emulsion, capsule, tablet, powder, granules, sprays, drops.

Preferably compositions may be formulated to be administered through the respiratory tract in a nebulizer, with or without propellants.

Such compositions can be prepared according to techniques and protocols known to the expert of the technical field. Said composition may even contain anti-inflammatory agents such as 18-beta glycyrrhetinic acid.

Object of the present invention are the compositions above described useful for treating infections of the upper respiratory tract, preferably for treating infections causing diseases such as acute otitis media, recurrence otitis media, nasal polyposis, sinusitis.

### EXAMPLES

### Collection of Nasal and Pharyngeal Swabs from Patients

Thirty one children aged between 10 and 12 years have been involved in this study. Children who had one, few or any AOM episode have been selected. Patients who received antibiotics in the previous two weeks, had an operation on the upper respiratory tract or with anatomic abnormalities of the respiratory tract have been excluded.

A nasal and pharyngeal swab has been collected respectively from the nostrils and the mouth of each patient with a cotton wool soaked in sterile calcium alginate.

### Microbiological Test

In order to highlight the presence of bacterial flora of nasal and pharyngeal swab samples were collected as above described, all samples have been plated onto Mitis Salivarius agar (Difco), a selective medium for streptococci, and onto “chocolate agar” (Columbia Agar Base, OXOID) containing 5% horse blood in order to determine bacterial microflora.

Cultures have been incubated for 18 hours at 37° C. in presence of 5% CO₂ and atmospheric pressure. All strains have been frozen at −70° C. in “Brain heart infusion broth” (OXOID) with 20% glycerol.

### BLIS (Bacteriocin-Like Inhibitory Substance) Test

Each colony morphologically distinct and isolated, obtained from the growth of bacteria as described above has been assayed for the ability to inhibit the most representative strains causing otitis: S. pyogenes 2812A, S. pneumoniae 11ATN, H. influenzae 3ATF, S. aureus, E. coli, P. aeruginosa, S. salivarius ATCC13419, M. catarrhalis. The ability to inhibit pathogen strains has been assayed by the “BLIS test” as originally developed by Walls et al. (Med microbial 52 (2003)). Assays have been performed by using two different media: Trypticase Soy Yeast Extract Calcium Agar (TSYCa)+2% Yeast Extract and Blood agar+calcium carbonate (BACa). Results have shown that the strain of Streptococcus salivarius identified by filing number DSM 23307 is able to inhibit the growth of S. pneumoniae both in TSYCa and BACa medium. Furthermore, it has been evaluated the ability of strain S. salivarius DSM 23307 to inhibit particularly virulent and multi-resistant strains of S. pneumoniae 19A and S. pyogenes M-type 1.

### Analysis of Virulence Genes

In S. salivarius DSM 23307 the presence of virulence genes particularly diffuse in streptococci such as sag A, smez-2 and speB, respectively responsible of the production of the toxin streptolisin S, the mitogenic exotoxin and the eritrogenic exotoxin. The assays have been performed by PCR and hybridization with specific probes.

In particular, total bacterial DNA was extracted in agarose plugs as previously described (Santagati et al., 2009). Following restriction with SacII enzyme (TaKaRa BIO), macro-restriction fragments were resolved in 1% agarose gel using 0.5× tris-borate-ethylene diamine tetra-acetic acid buffer (BioRad) at 14° C. The CHEF DR PFGE (BioRad) system was used, switch and run times were 1″ to 15″ for 20 hrs, with a voltage gradient of 6V/cm². The macro-restriction fragments were visualized by a blue-light trans-illuminator (Safe Imager Invitrogen) after staining with 1×SYBR Green (SYBR Safe DNA gel staining Invitrogen) in TBE 0.5×. The macro-restriction fragments were transferred from the gel to a nylon Hybond N+membrane (Amersham) using a Vacuum blotter 785 (BioRad) and denaturing solutions (NaOH 0.5M/NaCl 1.5M). DNA fragments were immobilized by UV irradiation (Ultraviolet Crosslinker, Amersham). The hybridization assay with sagA, smeZ-2, speB probes and further probes specific for spec, speJ, speG, prtF and sof were performed using the “ECL Direct Nucleic Acid Labelling and detection System” (RPN 3000 Amersham), following the protocol provided with the kit. The probes were obtained by PCR from the S. pyogenes SF370 and 2812A genome and purified with the QIAquick purification kit (Quiagen) using the primers listed in table 1.

TABLE 1
Primers used in this study

| | | | Amplicon | |
|---|---|---|---|---|
| | Primer | | size | Accession |
| Gene | name | Primer sequence (5′-3′) | (bp) | number |
| sivA | MS386 | TGATTTAACGCAACATCATCATCAACCTT | 640 | GQ857551 |
| | ^{(*)}MS387 | CATACATCAAATATCCATTCCTTG (SEQ | | |
| | | ID NO: 1) | | |
| nisA | MS389 | CAGGTGCATCACCACGCATTACAACCACT | 600 | DQ146939 |
| | ^{(*})MS390 | GAGTATCCAATCTTATACCC (SEQ ID | | |
| | | NO: 2) | | |
| nisF | MS391 | TGGAACAGTCTGTGGTTTATTAGGAGTCA | 560 | U17255 |
| | ^{(*})MS392 | CATTCCTCCATGCACAATCTTAA (SEQ | | |
| | | ID NO: 3) | | |
| NsaB | MS394 | ATCCTATGGTGAAGAGTATCGAGATCTAG | 774 | DQ146939 |
| | ^{(*)}MS395 | CCCCTAGATACTTAGAACAACC (SEQ | | |
| | | ID NO: 4) | | |
| mutII | MS396 | GGGTGGTAATCGTTGGTGGCAAGGCATAT | 560 | U40620 |
| | ^{(*})MS397 | CGCCTACTACTTGAAGGCTG (SEQ ID | | |
| | | NO: 5) | | |
| mutIiI | MS398 | TTGTGTCGCATGTCAGCGGCGGAGTTGAT | 664 | AF207710 |
| | ^{(*)}MS399 | CTCGTGTTATAACGGGC (SEQ ID NO: | | |
| | | 6) | | |
| sagA | MS380 | ATGTTAAAATTTACTTCAAATATTTTATT | 300 | CP000829 |
| | ^{(*})MS381 | ATTTACCTGGCGTATAACTTCCG (SEQ | | |
| | | ID NO: 7) | | |
| smeZ-2 | MS382 | GTAATTGATTTTAAAACCAGTCATAACGA | 400 | AF086626 |
| | ^{(*)}MS383 | TACTTTCTTTATCTCTATATCCTAC | | |
| | | (SEQ ID NO: 8) | | |
| speB | MS384 | GGCATGTCCGCCTACTTTACCGACAGGTG | 800 | NC_002797 |
| | ^{(*)}MS385 | CACGAAGCGCAGAAG (SEQ ID NO: | | |
| | | 9) | | |
| speC | MS410 | TACTGATTTCTACTATTTCACCTATCATC | 447 | NC_002737 |
| | ^{(*)}MS411 | TCTGATTTTAAAGTCAATTTCCTGG | | |
| | | (SEQ ID NO: 10) | | |
| speG | MS412 | GCTATGGAAGTCAATTAGCTTATGCAGCC | 488 | NC_007297 |
| | ^{(*)}MS413 | GATGTATAACGCGATTCCGA (SEQ ID | | |
| | | NO: 11) | | |
| speJ | MS414 | CACTCCTTGTACTAGATGAGGTTGCACGC | 508 | NC_007297 |
| | ^{(*)}MS415 | ATACGAAATCATACCAGTAG (SEQ ID | | |
| | | NO: 12) | | |
| prtF | MS400 | CGGAGTATCAGTAGGACATGCGGACTCCC | 882 | NC_011375 |
| | ^{(*})MS401 | ACCAACATTGCTTAATCCA (SEQ ID | | |
| | | NO: 13) | | |
| sof | MS402 | ATGCCTGGTTGGGTATCTTCGGTAGAGAA | 406 | NC_011375 |
| | ^{(*)}MS403 | CAAAACGTTCTGCGCCTA (SEQ ID | | |
| | | NO: 14) | | |

^{(*)}reverse primers
Results have shown the absence of such virulence genes.

### Adhesion Test

To perform the test cells HEp-2 (ATCC CCL 23) have been cultured in essential minimal Eagle media (EMEM) (Invitrogen, Carlsbad, Calif.). The media was added with 10% bovine serum (FBS), penicillin (100 U/ml) and streptomycin (100 μg/ml). Streptococcus salivarius bacteria DSM23307 before being used in the adhesion assay have been grown for 16-18 hours in a 5 ml Todd Hewitt media. Bacteria density has been adjusted according to spectrophotometer readings in order to have a range of density between 10⁵ and 10⁶ CFU/ml before the test. The adhesion test has been performed in HEp-2 cells as described in Benga L. et al. In particular, the resulting number of adherent bacteria was obtained by subtraction from the total number of CFU and expressed as percentage of adherence. All experiments were performed in duplicate wells and performed at least three times; wells containing only cells were used as controls. Bacterial adhesion to the HEp-2 cell layer was performed on microscope cover glasses according to the following protocol. Briefly, 2×10⁸ cells resuspended in PBS were incubated with a monolayer of HEp-2 cell for 1 h at 37° C. After washes with PBS, the cells were fixed with 3 ml of methanol and incubated for 8 min at room temperature. After removal of methanol, cells were stained with 3 ml of Giemsa stain solution (1:29, Carlo Erba, Milan Italy) for 30 min at room temperature. Wells were washed and then dried at 30° C. for 1 h. Adherent bacteria were examined microscopically (100× magnification) in 20 random microscopic fields obtaining count and average.

Adhesion indexes (ADI; number of bacteria/100 HEp-2 cells); strong adhesion: ADI>2500; good adhesion: 500<ADI<2500; weak adhesion: 100<ADI<500; no adhesion: ADI<100.

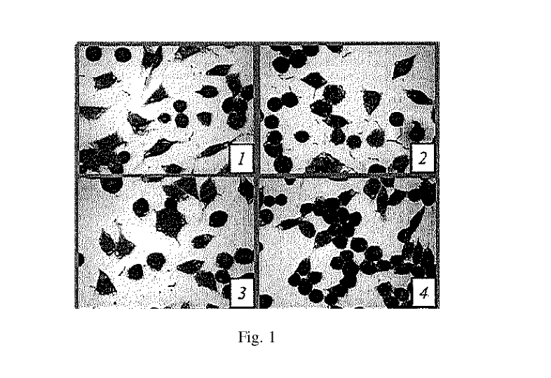

In the assay the ability to adhere to HEp-2 cell line of the S. salivarius strain DSM 23307 has been compared to that of strains S. salivarius K12 and S. salivarius 4SMB. The results were expressed as percentage adherence comparing the initial inoculum, the initial cell count (10⁶ CFU/ml) and the cells that adhered to HEp-2 cells after extensive washing with PBS. We found that between 50% and 57% of S. salivarius DSM 23307 remained attached to the HEp-2 monolayer, a similar percentage (50% to 60%) was found for S. salivarius K12, while S. salivarius 4SMB showed the lowest percentage of adhesion (25%-30%) (FIG. 1). The results on HEp-2 cell line adhesion was confirmed by microscopic examination. Therefore, the adhesion index of S. salivarius DSM 23307 and S. salivarius K12 (used as positive control) showed similar value of adhesion indicating good adhesion which can interfere with the adhesion of opportunistic bacteria and fungi on host cells.

Table 2 summarizes adhesion indexes found in the assay.

TABLE 2
Adhesion indexes

| | | AdI (Adhesion Index) |
|---|---|---|
| Species | Strain | Number of bacteria/100 FaDu cells) |
| S. salivarius | K12 | 1059 |
| S. salivarius | DSM 23307 | 1362 |
| S. salivarius | 4SMB | 500 |

### Stability Test

Stability tests have been performed by incubating the strain Streptococcus salivarius DSM 23307 for 18 hours at pH 8.0 in “Tryptic Soy” (TSB), Todd Hewitt and Brain Heart Infusion (BHI) media.

### Results

### Identification of Isolated Strain

In the table 3 below are identified the species to which belong the strain isolated from the analyzed nasal and pharyngeal swabs:

TABLE 3

| Bacteriocins producer | Molecular identification |
|---|---|
| 3A-TF(1) | S. mitis |
| 3A-TF(3) | S. salivarius |
| 8A-TF | S. mitis |
| 11A-TF(2) | S. mitis |
| 14A-TF(2) | S. salivarius |
| 14A-TF(4) | S. salivarius |
| 15A-TF | L. cremoris |
| 19A-TF(1) | S. sanguis |
| 21A-TF(3) | S. mitis |
| 24A-TF(4) | S. salivarius |
| 25A-TF | L. cremoris |
| 25A-TN(2) | S. salivarius |
| 26A-TF(1) | S. mitis |
| 25A-TF(2) | S. mitis |

Identification and Characterization of Strain Streptococcus salivarius DSM 23307

Streptococcus salivarius DSM 23307 has been isolated from the nosepharynx of a human subject. The strain growths on a “Mitis salivarius” medium at 35° C., 5% CO₂, having the typical morphology of S. salivarius species.

Colony shape and size: round, 1-2 mm diameter.

Edge: continuous, smooth.

Colour: Blue.

Grown on Columbia agar with 5% horse blood at 37° C., 5% CO₂ the strain is not haemolytic and has the following morphology

Colony shape and size: round, 1-2 mm diameter.

Edge: continuous, smooth.

Colour: White.

Streptococcus salivarius DSM 23307 strain has been analyzed by the commercial kit for the identification of streptococci API 20 Strep. After 24 hours incubation, according to the manufacturer's instruction, has resulted code 5070451, corresponding to the species Streptococcus salivarius.

Results obtained by API 20 Strep
Acetoin production: positive
Hydrolisis: negative
β-glucosidase: positive
Pirrolinodil arilamidase: negative
α-galactosidase: negative
β-glucuronidase: positive
Alcaline phosphatase: positive
Leucin arilamidase: positive
Arginine dihydrolase: negative
Ribose: negative
L-arabinose: negative
Mannitol: negative
Sorbitol: negative
Lactose: positive
Trealose: positive
Inuline: negative
Raffinose: negative
Glycogen: negative
B-hemolysis: negative

16S and sodA gene sequence analysis have demonstrated that the identified strain belongs to the species S. salivarius (99.8% identity).

Activity of S. salivarius DSM 23307

| | S. | S. | | | | |
|---|---|---|---|---|---|---|
| | pyogenes | pyogenes | S. pneu- | H. influ- | | |
| Incu- | 2812A M- | SF370 M- | moniae | enzae | S. | B. |
| bation | type 1 | type 1 | 11A-TN | 3A-TF | aureus | catarrhalis |
| BACa | − | − | + | − | − | − |
| TSYE | + | + | + | − | − | − |

Adhesion Experiment

Adhesion assays have demonstrated that the Streptococcus salivarius DSM 23307 strain has an excellent ability to adhere to HEp-2 cells, up to 57%, interfering with the adhesion of opportunistic bacteria and fungi.

Formulations
1. Streptococcus salivarius DSM 23307, saline.
2. Streptococcus salivarius DSM 23307, EG56 polymer, xanthan, carboxymethyl-cellulose, saline.
3. Streptococcus salivarius DSM2 3307, silicone, Vaseline, white soft, magnesium stearate.
4. Streptococcus salivarius DSM 23307, maltodextrin, mannitol, 18 beta-glycyrrhetinic acid, starch.
5. Streptococcus salivarius DSM 23307, glucose, deionized water.
6. Streptococcus salivarius DSM 23307, propylene glycol and/or glycerine.

In conclusion, the present invention provides a new bacterial strain belonging to the species Streptococcus salivarius having biological features making it the one and different from other patented strains indicated for the treatment of the above referred infections.

In particular, the strain Streptococcus salivarius DSM 23307 of the present invention inhibit even S. pneumoniae (the main pathogenic agent of AOM) in different culturing conditions (BACa and TSYE) and S. pyogenes (TSYE).

This feature differentiates it from other described strains belonging to S. salivarius such as S. salivarius 30 which has inhibitory ability only towards S. pyogenes in the two BACa and TSYE media, expanding its range of action only in assays carried out in TSYE.

Furthermore, surprisingly as demonstrated by BLIS tests results, the strain Streptococcus salivarius DSM 23307 of the present invention inhibits even the important pathogens such as S. pneumoniae 19A and S. pyogenes M-type 1, which are frequently isolated from the upper respiratory tracts.

Finally, S. salivarius DSM 23307, shows some biological features, such as sensitivity to antibiotics, absence of virulence genes, and adhesive ability up to 57%, which make it the one, well characterized and distinguishable from other S. salivarius strains, in particular S. salivarius 30.

### REFERENCES

1. Altschul S F, Madden T L, Schäffer A A, Zhang J, Zhang Z, Miller W, Lipman D J. 1997. Gapped BLAST and PSI-BLAST: a new generation of protein database search programs. Nucleic Acids Res. 1;25(17):3389-402.
2. Aas J A, Paster B J, Stokes L N, Olsen I, Dewhirst F E. 2005. Defining the normal bacterial flora of the oral cavity. J Clin Microbiol. 43(11):5721-32.
3. Benga L, Goethe R, Rohde M, Valentin-Weigand P. 2004. Non-encapsulated strains reveal novel insights in invasion and survival of Streptococcus suis in epithelial cells. Cell Microbiol. 6(9):867-81.
4. Caglar E, Kargul B, Tanboga I. 2005. Bacteriotherapy and probiotics' role on oral health. Oral Dis. 11(3):131-7.
5. Falagas M E, Rafailidis P I, Makris G C. 2008. Bacterial interference for the prevention and treatment of infections. Int J Antimicrob Agents. 31(6):518-22.
6. Goldstein B P, Wei J, Greenberg K, Novick R. 1998. Activity of nisin against Streptococcus pneumoniae, in vitro, and in a mouse infection model. J Antimicrob Chemother. 42(2):277-8.
7. Hynes W L, Ferretti J J, Tagg J R. 1993. Cloning of the gene encoding Streptococcin A-FF22, a novel lantibiotic produced by Streptococcus pyogenes, and determination of its nucleotide sequence. Appl Environ Microbiol. 59(6):1969-71.
8. Karaya K, Shimizu T, Taketo A. 2001. New gene cluster for lantibiotic streptin possibly involved in streptolysin S formation. J. Biochem. 129(5):769-75.
9. Marsh P D. 1991. The significance of maintaining the stability of the natural microflora of the mouth. Br Dent J. 171(6):174-7.
10. Power D A, Burton J P, Chilcott C N, Dawes P J, Tagg J R. 2008. Preliminary investigations of the colonisation of upper respiratory tract tissues of infants using a paediatric formulation of the oral probiotic Streptococcus salivarius K12. Eur J Clin Microbiol Infect Dis. 27(12):1261-3.
11. Santagati M, Lupo A, Scillato M, Di Martino A, Stefani S. 2008. Conjugal mobilization of the mega element carrying mef(E) from Streptococcus salivarius to Streptococcus pneumoniae. FEMS Microbiol Lett. 290(1):79-84.
12. Saunders K A, Ball L C. 1980. The influence of the composition of blood agar on beta haemolysis by Streptococcus salivarius. Med Lab Sci. October; 37(4):341-5.
13. Tagg J R, Bannister L V. 1979. “Fingerprinting” beta-haemolytic streptococci by their production of and sensitivity to bacteriocine-like inhibitors. J Med. Microbiol. 12(4):397-411.
14. Upton M, Tagg J R, Wescombe P, Jenkinson H F. 2001. Intra- and interspecies signaling between Streptococcus salivarius and Streptococcus pyogenes mediated by SalA and SalA1 lantibiotic peptides. J. Bacteriol. July; 183(13):3931-8.
15. Walls T, Power D, Tagg J. 2003. Bacteriocin-like inhibitory substance (BLIS) production by the normal flora of the nasopharynx: potential to protect against otitis media? J Med. Microbiol. 52(Pt 9):829-33.
16. Wescombe P A, Burton J P, Cadieux P A, Klesse N A, Hyink O, Heng N C, Chilcott C N, Reid G, Tagg J R. 2006. Megaplasmids encode differing combinations of lantibiotics in Streptococcus salivarius. Antonie Van Leeuwenhoek. 90(3):269-80.
17. Wescombe P A, Heng N C, Burton J P, Chilcott C N, Tagg J R. 2009. Streptococcal bacteriocins and the case for Streptococcus salivarius as model oral probiotics. Future Microbiol. 4(7):819-35.
18. Wirawan R E, Klesse N A, Jack R W, Tagg J R. 2006. Molecular and genetic characterization of a novel nisin variant produced by Streptococcus uberis. Appl Environ Microbiol. 72(2):1148-56.
19. Zheng J, Liu G, Zhu W, Zhou Y, Liu S. 2004. Phylogenetic clusters of rhizobia revealed by genome structures. Sci China C Life Sci. 47(3):268-78.

1. A pure bacterial strain belonging to the species S. salivarius deposited at the Deutsche Sammlung von Mikroorganismen and Zellkulturen GmbH, Braunschweig, Germany, under accession number DSM 23307.

2. The bacterial strain according to claim 1 useful for the treatment of upper respiratory tract infections and/or inflammation.

3. The bacterial strain according to claim 1 useful for the treatment of infections which are cause of diseases including acute otitis media, recurrent otitis media, sinusitis and or conditions characterized by inflammation such as nasal polyposis.

4. The bacterial strain according to claim 1 wherein the bacteria are in suspension, freeze-dryed or inactivated, provided they are not killed.

5. Compositions comprising the bacterial strain according to claim 1 useful for the treatment of upper respiratory tract infections and/or inflammation.

6. Compositions comprising the bacterial strain according to claim 1 useful for the treatment of infections which are cause of diseases including acute otitis media, recurrent otitis media, sinusitis and or conditions characterized by inflammation such as nasal polyposis.

7. Compositions according to claim 5 implemented by freeze-drying of bacterial culture, by mixing freeze-dryed bacteria both in suspension with water or with further suitable excipients and optionally with addition of further active principles.

8. Composition according to claim 5 wherein the amount of bacteria is preferably in the range between 10³ and 10¹⁰ CFU for each gram of composition.

9. Compositions according to claim 5 comprising one or more pharmaceutically acceptable excipients, aromatizing agents or carriers.

10. Compositions according to claim 9 wherein the used excipients are: rubber, xanthan, carboxylmethyl cellulose, silicone, Vaseline, white soft, magnesium stearate, maltodextrin, mannitol, starch, glucose, glycerine, propylene glycol, and equivalent molecules.

11. Compositions according to claim 9 wherein the used carriers are idoneous to improve the bioavalibility, the stability and the endurance of the microrganism.

12. Compositions according to claim 9 wherein the used carriers improve the adhesion of the microrganism on the mucosal surface such as the Bis-Methoxy PEG-13 PEG-438/PPG-110 SMDI copolymer.

13. Compositions according to claim 5 comprising anti-inflammatory agents such as 18-beta glycyrrhetinic acid.

14. Compositions according to claim 5 characterized in being in any form suitable to be administered topically, orally or through the respiratory tract.

15. Compositions according to claim 14 characterized in being in the pharmaceutical form of cream, lotion, gel, ointment, solution, suspension, emulsion, capsule, tablet, powder, granules, sprays, drops.

16. Compositions according to claim 15 characterized in being formulated to be administered through the respiratory tract in a nebulizer, with or without propellants


SEQUENCE LISTING (14 sequences)

SEQ ID NO: 1 - DNA, 53 bp, Artificial Sequence
  Sythetic Primer
tgatttaacgcaacatcatcatcaaccttcatacatcaaatatccattccttg

SEQ ID NO: 2 - DNA, 49 bp, Artificial Sequence
  Sythetic Primer
caggtgcatcaccacgcattacaaccactgagtatccaatcttataccc

SEQ ID NO: 3 - DNA, 52 bp, Artificial Sequence
  Synthetic Primer
tggaacagtctgtggtttattaggagtcacattcctccatgcacaatcttaa

SEQ ID NO: 4 - DNA, 51 bp, Artificial Sequence
  Synthetic Primer
atcctatggtgaagagtatcgagatctagcccctagatacttagaacaacc

SEQ ID NO: 5 - DNA, 49 bp, Artificial Sequence
  Synthetic Primer
gggtggtaatcgttggtggcaaggcatatcgcctactacttgaaggctg

SEQ ID NO: 6 - DNA, 46 bp, Artificial Sequence
  Synthetic Primer
ttgtgtcgcatgtcagcggcggagttgatctcgtgttataacgggc

SEQ ID NO: 7 - DNA, 52 bp, Artificial Sequence
  Synthetic Primer
atgttaaaatttacttcaaatattttattatttacctggcgtataacttccg

SEQ ID NO: 8 - DNA, 54 bp, Artificial Sequence
  Synthetic Primer
gtaattgattttaaaaccagtcataacgatactttctttatctctatatcctac

SEQ ID NO: 9 - DNA, 44 bp, Artificial Sequence
  Synthetic Primer
ggcatgtccgcctactttaccgacaggtgcacgaagcgcagaag

SEQ ID NO: 10 - DNA, 54 bp, Artificial Sequence
  Synthetic Primer
tactgatttctactatttcacctatcatctctgattttaaagtcaatttcctgg

SEQ ID NO: 11 - DNA, 49 bp, Artificial Sequence
  Synthetic Primer
gctatggaagtcaattagcttatgcagccgatgtataacgcgattccga

SEQ ID NO: 12 - DNA, 49 bp, Artificial Sequence
  Synthetic Primer
cactccttgtactagatgaggttgcacgcatacgaaatcataccagtag

SEQ ID NO: 13 - DNA, 48 bp, Artificial Sequence
  Synthetic Primer
cggagtatcagtaggacatgcggactcccaccaacattgcttaatcca

SEQ ID NO: 14 - DNA, 47 bp, Artificial Sequence
  Synthetic Primer
atgcctggttgggtatcttcggtagagaacaaaacgttctgcgccta